(19) United States
(12) Patent Application Publication
(10) Pub. No.: US 2024/0188943 A1
(43) Pub. Date: Jun. 13, 2024

(54) A Collector and an Intermittent Urinary Catheter Assembly comprising a Collector

(71) Applicant: Coloplast A/S, Humlebaek (DK)

(72) Inventor: Troels Gottfried Pedersen, Gilleleje (DK)

(21) Appl. No.: 18/286,775

(22) PCT Filed: Apr. 13, 2022
(86) PCT No.: PCT/DK2022/050079

§ 371 Date: Oct. 13, 2023

(30) Foreign Application Priority Data
Apr. 15, 2021 (DK) PA 2021 70174

(51) Int. Cl. A61B 10/00; A61M 25/00

(52) U.S. Cl. CPC A61B 10/007; A61M 25/0017

(57) ABSTRACT

A urine sample collector configured for collecting a sample of urine during catherization with a urinary catheter, the collector has a proximal end and a distal end, the collector comprising an inlet positioned at the proximal end and an outlet configured for drainage of fluid positioned at the distal end, the collector comprising a lumen extending in longitudinal direction of the collector from the inlet to the outlet along a central longitudinal axis (A), the lumen forming a channel configured for transport of fluid from the inlet to the outlet, the collector comprising an outer wall, an inner wall and a distal end, the outer wall encircling the inner wall, the inner wall defining an inner volume at the distal part of the collector corresponding to a distal part of the lumen, the outer wall comprising at least a distal outer wall portion and a proximal outer wall portion, and the inner wall comprises a distal inner wall portion, the distal outer wall portion, the distal inner wall portion and the distal end defines a collecting chamber, wherein the collecting chamber comprises one or more chamber inlet ports, the one or more chamber inlet ports being defined by a clearance between the outer wall and the inner wall.

The invention relates to a collector and an intermittent urinary catheterisation assembly comprising a collector.

### BRIEF DESCRIPTION OF THE DRAWING

The accompanying drawings are included to provide a further understanding of embodiments and are incorporated into and a part of this specification. The drawings illustrate embodiments and together with the description serve to explain principles of embodiments. Other embodiments and many of the intended advantages of embodiments will be readily appreciated as they become better understood by reference to the following detailed description. The elements of the drawings are not necessarily to scale relative to each other. Like reference numerals designate corresponding similar parts.

FIG. 1 illustrates a schematic cross-sectional side view of a collector,

FIG. 2 illustrates a schematic cross-sectional side view of another collector,

FIG. 3 illustrates a schematic cross-sectional side view of an embodiment of a collector,

FIG. 4 illustrates two schematic cross-sectional end views of two embodiments of the collector illustrated in FIG. 3,

FIG. 5 illustrates a schematic cross-sectional side view of an embodiment of a collector,

FIG. 6 illustrates a schematic cross-sectional side view of an embodiment of a collector and a distal part of an intermittent urinary catheter.

### DETAILED DESCRIPTION

Embodiments, and features of the various exemplary embodiments described in this application, may be combined with each other (“mixed and matched”), unless specifically noted otherwise.

A urine sample is one of the most common health tests. Urine contains waste products that are filtered out of the body. If a urine sample contains anything unusual, it may indicate an underlying health problem, and by examining the waste products that are excreted by the urine, it is possible to gain knowledge about the body's functions or imbalances.

Urine tests are commonly done to check for infections—such as a urinary tract infection (UTI). A urine sample can be examined by a doctor or in a laboratory for content such as protein (egg white), blood and sugar. If these tests are positive, it may be due to kidney or urinary tract disease or diabetes, and may indicate that further examination is needed. A urine sample can help a user and/or healthcare professional to diagnose or rule out certain health conditions.

Urinary tract infection (UTI) can be a problem especially for some users of intermittent catheters. Intermittent catherization requires the insertion of a foreign object (a catheter) into the urethra to drain the bladder. This may increase the possibility of bacteria being pushed further into the urethra, also called catheter-related urinary tract infection. Additionally, UTI signs and symptoms can be difficult for some IC users to interpret, such as users with spinal cord injury, as they do not feel pain. Therefore, it is desirable to discover infections as early as possible. Typically, a urine sample can be taken by urinating in a small container or by collecting urine in a collecting bag connected to an intermittent catheter and then afterwards a urine sample can be extracted from the collecting bag.

However, there is still need for a more convenient solution for catheter users.

Embodiments relate to a urine sample collector configured for collecting a sample of urine during catherization with a urinary catheter, where the collector has a proximal end and a distal end, the collector comprising an inlet positioned at the proximal end and an outlet configured for drainage of fluid positioned at the distal end, the collector comprising a lumen extending in longitudinal direction of the collector from the inlet to the outlet along a central longitudinal axis (A), the lumen forming a channel configured for transport of fluid from the inlet to the outlet, the collector comprising an outer wall, an inner wall and a distal end, the outer wall encircling the inner wall, the inner wall defining an inner volume at the distal part of the collector corresponding to a distal part of the lumen, the outer wall comprising at least a distal outer wall portion and a proximal outer wall portion, and the inner wall comprising a distal inner wall portion, where the distal outer wall portion, the distal inner wall portion and the distal end defines a collecting chamber, wherein the collecting chamber comprises one or more chamber inlet ports, the one or more chamber inlet ports being defined by a clearance between the outer wall and the inner wall.

A collector as described above allows a user to collect a sample of urine during intermittent-catherization. The collection of a sample of urine is done simultaneously as drainage of the bladder by the catheter. A user does not need to interrupt the drainage of the bladder to take a sample of urine or try to fill a small container. The drainage flow rate is unaffected by the collector. The construction of the collector allows a sample to be taken without any undesirable dripping or spillage.

Whenever referring to the proximal end of the collector, the referral is to the end closest to the catheter. Likewise, whenever referring to the distal end of the collector, the referral is to the end furthest away from the catheter. In other words, the proximal end is the end closest to the user, when a catheter is to be inserted and the distal end is the opposite end, the end furthest away from the user, when the catheter is to be inserted. The same definitions apply to the collector.

The longitudinal direction is the direction from the distal end to the proximal end of the collector. The transverse direction is the direction perpendicular to the longitudinal direction, which corresponds to the direction across the collector.

The axial direction and the longitudinal direction of the collector extends from the inlet to the outlet. Radial direction is the direction transverse to the axial direction.

The catheter described in this application is used as a urinary catheter, such as an intermittent urinary catheter.

The catheter may comprise a main tubular part extending from the distal end to the proximal end. The tip is positioned in the proximal end of the catheter and is provided as a rounded closed end of the tube constituting the main part of the catheter. The catheter may comprise a connector in the distal end and may in an embodiment comprise a flared end of the catheter so that the diameter of the connector increases with respect to the tubular part. The catheter may also comprise a handle in the distal end, which has a length allowing the user to manipulate the catheter.

The collector may be a part of the connector and be an integrated part of the catheter.

Intermittent catheters are typically inserted by the user him- or herself and sits only in the urethra and bladder for as long as it takes to empty the bladder—e.g. for about 5-10 minutes. Intermittent catheters are commonly used every 4-6 hours to empty the bladder corresponding roughly to the interval that people having no urinary problems will usually go to the bathroom. Intermittent catheters are typically more rigid than indwelling catheters since they have to be inserted by the user him-/herself and since they do not need to sit in the urethra for days or weeks. An important feature for the intermittent catheter is to ease the insertion into the urethra. This is done by providing the intermittent catheter with a low frictious surface. Non-limiting examples of such are hydrophilic coated catheters which are subsequently wetted by a swelling media in order to produce a low friction surface, or oil or water-based gel which is applied to the catheter before insertion into the urethra.

Intermittent urinary catheters may be provided with a hydrophilic coating that needs to be wetted prior to use and thereby absorbs a considerable amount of liquid. Such a hydrophilic coating will provide a very lubricious surface that has very low-friction when the catheter is to be inserted. Hydrophilic coated catheters, where the coating absorbs a considerable amount of liquid for a low frictious surface (swelling degree >100%), will not be suitable for indwelling catheters, because the hydrophilic surface coating would stick inside the mucosa of the urethra if left inside the body for a longer period, due to the hydrophilic coating transforming from being highly lubricious when fully wetted to being adhesive when the hydration level of the coating is reduced.

The intermittent urinary catheters may be provided with a hydrophilic coating of the kind that is wetted prior to use to absorb a considerable amount of liquid and to provide a very lubricious surface.

Usually, catheters used as urinary draining devices are from size 8 FR to size 18 FR. FR (or French size or Charriere (Ch)) is a standard gauge for catheters approximately corresponding to the outer circumference in mm. More accurately, the outer diameter of the catheter in mm corresponds to FR divided by 3. Thus, 8 FR corresponds to a catheter with an outer diameter of 2.7 mm and 18 FR corresponds to a catheter with an outer diameter of 6 mm.

Intermittent urinary catheters typically range from CH 8-CH 16.

Intermittent urinary catheters are predominantly used for self-catheterisation by the user. In one embodiment, the intermittent urinary catheter is a telescopic catheter.

The hydrophilic coating may be provided only on the insertable part of the catheter. The hydrophilic surface coating is of the kind which, when hydrated or swelled using a swelling medium, reduces the friction on the surface area of the catheter which is intended to be inserted into the urinary passage of a user corresponding to the insertable part of the catheter.

The catheter package may comprise a medium for activating the hydrophilic surface coating of the catheter. The activating medium may be a water-based substance, such as sterile water, saline-solution, or any water-based liquid. Furthermore, the activating medium may be in the form of a vapour contributing material, such as a wetted sponge, woven or non-woven material comprising a vapour contributing liquid. By introducing a vapour contributing material into the package, the vapour will over time hydrate the hydrophilic coating ensuring that the coating is activated and that the hydrophilic coating provides a low-friction surface for the catheter.

In embodiments, one or more chamber inlet ports comprise an annular shaped opening or one or more openings shaped as a sector of an annular shape.

In embodiments, the distal outer wall portion and the distal inner wall portion extend co-axially in the longitudinal direction along the central longitudinal axis A of the collector.

In embodiments, the one or more chamber inlet ports are radially offset from the central longitudinal axis (A), and the one or more chamber inlet ports extend in a radial direction between the inner wall and the outer wall and are positioned facing the lumen in longitudinal direction towards the proximal part of the collector.

In embodiments, the one or more chamber inlet ports are positioned in radially direction from the central longitudinal axis A in a distance of 2 mm, 3 mm, 4 mm or 5 mm or more.

In embodiments, the inner wall comprises a top wall, which extends outwardly in radial direction towards the outer wall, where the radial extend of the one or more chamber inlet ports is defined by the clearance between the top wall and the outer wall in the radial direction.

Hereby is achieved that the collecting chamber is filled at low flow rate/velocity and as an effect the collected fluid in the collecting chamber is not flushed and replaced by a new sample of urine during the catheterization. The extend of the top wall decreases the risk of spillage from the collecting chamber when the collector is handled by a user.

In embodiments, the outer wall comprises a top wall portion, which extends inwardly in radial direction towards the inner wall, where the size of the one or more chamber inlet ports is defined by the clearance between the top wall portion and the inner wall in the radial direction.

In embodiments, the clearance at the one or more chamber inlet ports between the outer wall and the inner wall in the radial direction is 1.4 mm or more.

Embodiments relate to the one or more chamber inlet ports in a radially direction being narrower than the collecting chamber.

In embodiments, the proximal outer wall portion comprises a cylinder-shaped inner surface, which defines an inner volume at the proximal part of the collector corresponding to a proximal part of the lumen.

In embodiments, the inner wall comprises a cylinder-shaped proximal inner wall portion defining an inner volume at an intermediate portion of the lumen, where the intermediate portion of the lumen interconnects the proximal portion of the lumen and the distal portion of the lumen, the proximal portion and the intermediate portion and the distal portion of the lumen forming a through-going channel for providing flow of liquid through the collector.

In embodiments, the proximal inner wall portion comprises one or more side drainage openings allowing flow of fluid from a volume outside the proximal inner wall portion and radially inwards into the intermediate portion of the lumen.

In embodiments, the collector comprises at least one end opening at the proximal end of the proximal inner wall portion, where the at least one end opening extends in radial and or axial direction adapted to allow passage of fluid into the intermediate portion of the lumen through the proximal end or side wall of the proximal inner wall portion. The end opening may be arranged in the flow restrictor.

In embodiments, the collector comprises a flow restrictor configured for partially or completely restrict fluid flow, the flow restrictor being positioned at the proximal end of the proximal inner wall portion. Hereby, the flow restrictor will interrupt the flow path and the flow pattern in the proximal portion of the collector lumen will change. Portions of the fluid will be directed towards the one or more inlet ports.

The minimum cross-sectional area of the through-going passage of the flow restrictor may be in the range of 17-95% of the size of the first maximum cross-sectional area of the internal lumen of the urinary catheter.

The cross-sectional area of the through-going passage of the flow restrictor may be in the range of 17-95% of the size of the first maximum cross-sectional area of the internal lumen of the intermittent urinary catheter.

In embodiments, the collector comprises at least one end opening at the proximal end of the proximal inner wall portion, the at least one end opening extends in radially and/or in axially direction adapted to allow passage of fluid into the intermediate portion of the lumen through the proximal end or side wall of the proximal inner wall portion.

The end opening may extend radially, axially or in both radial and axial direction such that it allows passage of fluid into the intermediate part of the lumen through the flow restrictor and through the proximal inner wall.

The flow restrictor and the at least one end opening changes the flow path downstream through the collector, such as to force some fluid in direction of the collector chamber.

In embodiments, the sum of the cross-sectional area of the one or more side drainage openings and the at least one end opening is equal to or larger than the smallest cross-sectional area of the inlet. These openings may be so proportioned that the total cross-sectional areas are always equal to or greater than the cross-sectional area of the inlet, such that flow through collector lumen is not reduced.

The lumen may comprise a proximal, an intermediate and a distal portion.

In embodiments, the lumen comprises a varying cross-sectional area in a radial direction, where the smallest size of the cross-sectional area is equal to the size of the cross-sectional area of the inlet or larger.

In embodiments, the one or more chamber inlet ports comprise a size in transverse direction in the range of 20-60% of the size of the largest size of the distal outer wall portion. In an embodiment, the outer wall may have a diameter of 15 mm, such as 12-20 mm.

In embodiments, the collecting chamber is divided into two or more sub-chambers. The sub-chambers allow for different analysis scenarios.

In one embodiment, the sub-chambers may be filled sequentially or almost sequentially, so that one sub-chamber is filled before another sub-chamber is filled. Thus, urine may react chemically with a substance in the first sub-chamber before continuing to a second sub-chamber for a further analysis or chemical reaction.

In other embodiments the sub-chambers may be filled simultaneously—or almost simultaneously—allowing for collecting urine across a wider range of urine drainage. As an example, one sub-chamber may be filled within few seconds after voiding has started, whereas other sub-chamber(s) may be filled over a longer period of time, collecting a more average urine sample.

In embodiments, the outer wall may comprise a gateway that allows for extract of a urine sampling from the chamber or from one or mores sub-chambers in case a urine sample from the collector is desirable.

In embodiments, the collector comprises a gateway or port for extracting a sample of urine from the collecting chamber. The outer wall may comprise a gateway and/or port that allows for e.g. a syringe needle or sampling needle to penetrade the gateway and enable sampling of urine from the chamber in the collector. The gateway may be formed by a material that differs from the out wall/collector material in general. As example, the gateway may be made from a piece of rubber—natural or polymer, or simply an area of the outer wall that is so thin that peneration with the needle is feasible. The proximal outer wall portion may comprise a shape of a truncated cone, the inlet port of the collector is positioned at the proximal end, such as frusto-conical shape, such as part of the truncated cone.

The proximal outer wall portion may comprise a first type of one or more coupling parts adapted to connect the inlet port of the collector to the distal end of a catheter lumen. The proximal outer wall portion and the distal outer wall portion may comprise a second type of complementary shaped coupling parts adapted to open the one or more inlet ports. The distal outer wall portion and the proximal outer wall portion may comprise a second type of complementary shaped coupling parts adapted to open the one or more inlet ports.

The outlet may comprise a connector or a rim extending in longitudinal direction.

In embodiments, the distal outer wall portion comprises a see-through portion adapted for allowing a user to see through the outer wall.

In embodiments, the collecting chamber comprises a test pad. The test pad may be a chemical and/or optical test. The distal outer wall portion may comprise a weakened wall portion allowing a pellet or chemical compound/substance to be pressed into the collecting chamber. Hereby, a user may press the pellet or chemical compound/substance into the collecting chamber prior to collecting a urine samble.

Embodiments relate to an intermittent urinary catheterisation assembly for self-catheterisation comprising:
a catheter tube comprising an insertable portion intended for insertion into a user's urethra, where the catheter tube comprises a catheter lumen, a non-insertable portion not intended for insertion into the user's urethra, and a connecting portion being integral with or mounted to the non-insertable portion; the intermittent urinary catheterisation assembly comprising a collector as disclosed. The assembly may comprise a catheter tube, wherein at least an outer surface of the insertable portion of the catheter tube comprises a hydrophilic surface coating.

The inner lumen of the urine sample collector and the catheter lumen of the catheter tube may be so proportioned such that the cross-sectional area of the inner lumen of the collector is equal to or greater than the cross-sectional area of the catheter lumen of the catheter.

The assembly may comprise complementary shaped coupling parts to allow attachment and detachment of the coupling of the connector to the distal end of the inner lumen. The inner lumen of the collector is a continuation of the catheter lumen allowing drainage of a bladder through the catheter and the collector.

In embodiments, a urine sample collector configured for collecting a sample of urine during catherization with a urinary catheter, the collector having a proximal end and a distal end, the collector comprises an inlet positioned at the proximal end and an outlet positioned at the distal end configured for drainage of fluid, the collector comprising a lumen extending in longitudinal direction of the collector from the inlet to the outlet along a central longitudinal axis (A), the lumen forming a channel configured for transport of fluid from the inlet to the outlet, the collector comprising an outer wall, an inner wall and a distal end, the outer wall encircling the inner wall, the inner wall defining an inner volume at the distal part of the collector corresponding to a distal part of the lumen, the outer wall comprising at least a distal outer wall portion and a proximal outer wall portion, and the inner wall comprising a distal inner wall portion, wherein the distal outer wall portion, the distal inner wall portion and the distal end defines a collecting chamber, and wherein the collecting chamber comprises one or more chamber inlet ports, the one or more chamber inlet ports are extending radially facing the proximal end of the collector.

Hereby, fluid may flow into the chamber in a direction substantially parallel to the central longitudinal axis (A).

A collector as described above allows a user to collect a sample of urine during intermittent-catherization. The collection of the sample of urine is done simultaneously as the drainage of the bladder by the catheter and the drainage flow rate is unaffected by the collector. A user does not need to interrupt the drainage of the bladder to take a sample of urine. The construction of the collector allows a sample to be taken without any undesirable dripping or spillage.

Various exemplary embodiments and details are described hereinafter, with reference to the figures when relevant. It should be noted that the figures may or may not be drawn to scale and that elements of similar structures or functions are represented by reference numerals throughout the figures. It should also be noted that the figures are only intended to facilitate the description of the embodiments. They are not intended as an exhaustive description of the invention or as a limitation on the scope of the invention. In addition, an illustrated embodiment needs not have all the aspects or advantages shown. An aspect or an advantage described in conjunction with a particular embodiment is not necessarily limited to that embodiment and can be practiced in any other embodiments even if not illustrated, or if not explicitly described.

Embodiments provide an intermittent urinary catheter assembly comprising a collector that enable a sample of urine to be collected during catheterisation.

### DETAILED DESCRIPTION OF THE DRAWING

Embodiments, and features of the various exemplary embodiments described in this application, may be combined with each other (“mixed and matched”), unless specifically noted otherwise.

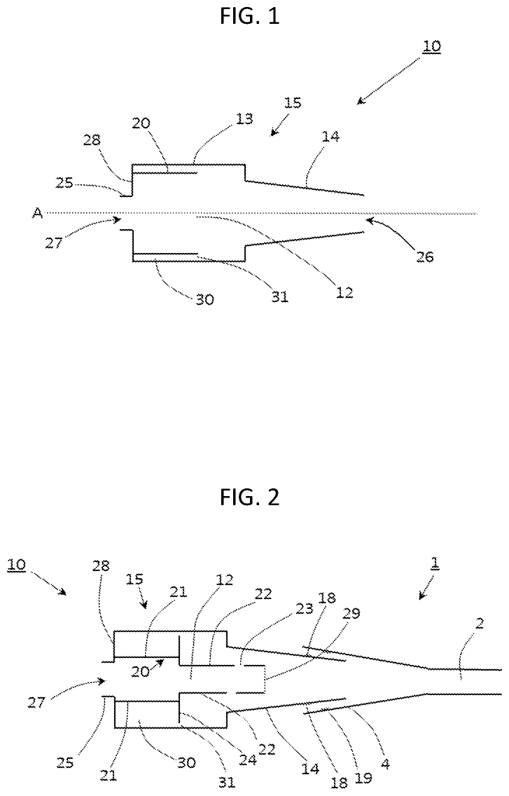

FIG. 1 illustrates a schematic cross-sectional view of a collector 10 configured for collecting a urine sample during catherization with a catheter, such as an intermittent urinary catheter.

The collector has a proximal end and a distal end. The collector 10 comprises an inlet 26 positioned at the proximal end and an outlet 27 positioned at the distal end. The collector comprises a lumen 12 extending in longitudinal direction of the collector from the inlet 26 to the outlet 27 along a central longitudinal axis (A). The lumen 12 forms a channel configured for transport of fluid from the inlet to the outlet 27. The outlet may comprise a connector or a rim 25 extending in longitudinal direction.

The collector comprises an outer wall 15, an inner wall 20 and a distal end 28.

The outer wall encircles the inner wall. The cylinder-shaped inner wall defines an inner volume at the distal part of the collector 10 corresponding to a distal part of the lumen 12.

The distal end 28 extends radially and connects the cylinder-shaped inner wall 20 and the cylinder-shaped outer wall 15 forming a collecting chamber 30.

The inner wall 20 and the outer wall 15 are arranged co-axially. The clearance between the inner wall 20 and the outer wall 15 defines an annularly shaped inlet port 31 to the collecting chamber 30.

The outer wall 15 comprises a distal outer wall portion 13 and a proximal outer wall portion 14. The proximal outer wall portion 14 is illustrated as a wall having a frusto-conical shape.

FIG. 2 illustrates schematically the collector and a distal part of an intermittent urinary catheter 1, such as a connector. The catheter 1 comprises a catheter tube with a catheter lumen 2. The collector 10 has an inner wall 20, an outer wall 15, a distal end 28 and an inner lumen 12 extending from the inlet to the outlet 27. The inner lumen 12 of the collector is a continuation of the catheter lumen 2 adapted to drain fluid from the tip of the catheter and out from the collector outlet. The collector 10 may be detachably attached to or a fixed part of the catheter 1.

The inner wall 20 comprises a distal inner wall portion 21, a proximal inner wall portion 22 and a top wall 24 extending radially outwards. The collecting chamber 30 is defined by the outer wall 15, the distal inner wall portion 21 and the radially outwardly extending top wall 24. Fluid may flow into the collecting chamber through the annularly shaped inlet port 31. The size and shape of the chamber inlet port 31 is defined by the small clearance between the top wall 24 and the outer wall 15.

The proximal inner wall portion 22 comprises drainage openings 23, which allows flow of fluid through the collector from a volume at the proximal end outside the proximal inner wall portion 22 and radially inwards into the intermediate portion of the lumen 12.

The collector 10 comprises a flow restrictor 29. The flow restrictor 29 may partially or completely block the proximal end of the proximal inner wall portion 22.

The outer wall 15 comprises the proximal outer wall 14 portion having a frusto-conical shape. The flow restrictor 29 is arranged within the volume defined by the proximal outer wall portion 14.

The drainage openings 23 have a size equal or larger than the inlet of the collector to avoid decrease in flow rate through the collector. The sum of the cross-sectional area of the one or more side drainage openings and the at least one end opening is equal to or larger than the smallest cross-sectional area of the inlet.

The assembly of collector and catheter comprises complementary shaped coupling parts 18,19 to allow attachment and detachment of the coupling of the connector to the distal end of the catheter.

The complementary shaped coupling parts 18,19 may form a connection, such as a threaded connection or snap fit connection.

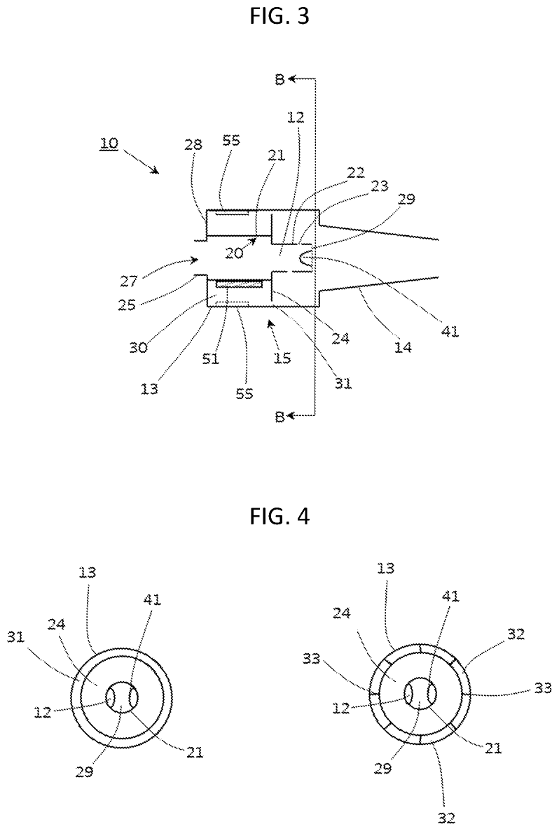

FIG. 3 illustrates the collector 10 comprising an inner wall 20, an outer wall 15, a distal end 28 and an inner lumen 12 extending from the inlet to the outlet 27.

The inner wall 20 comprises a distal inner wall portion 21, a proximal inner wall portion 22 and a top wall 24 extending radially outwards. The collecting chamber 30 is defined by the outer wall 15, the distal end 28, the distal inner wall portion 21 and the radially outwardly extending top wall 24. Fluid may flow into the collecting chamber through the inlet port 31.

The size and shape of the chamber inlet port 31 is defined by the clearance between the top wall 24 and the outer wall 15.

The proximal inner wall portion 22 comprises drainage openings 23 allowing flow of fluid through the collector from a volume outside the proximal inner wall portion 22 and radially inwards into the intermediate portion of the lumen 12. The collector 10 comprises a flow restrictor 29. The flow restrictor 29 may partially or completely block the proximal end of the proximal inner wall portion 22. In FIG. 3 the flow restrictor 29 comprises an end opening 41. Therefore, the flow restrictor 29 is partially blocking the proximal end of the proximal inner wall portion 22.

The outer wall 15 comprises a cylinder-shaped distal outer wall portion 13 and a truncated cone-shaped proximal outer wall 14 portion. The flow restrictor 29 is arranged within the volume defined by the distal outer wall portion 13.

The drainage openings 23 have a size equal to or larger than the inlet of the collector to avoid decrease in flow rate through the collector. The sum of the cross-sectional area of the one or more side drainage openings and the at least one end opening is equal to or larger than the smallest cross-sectional area of the inlet.

The collecting chamber 30 comprises a test pad 51. The distal outer wall 13 comprises a see-through portion 55 adapted for a user to see through the outer wall 15 and into the collecting chamber 30. The test pad may be of the kind changing colour when it is exposed for urine in the collecting chamber. The user may see the colour change of the test pad 51 through the see-through portion 55.

The cross-sectional view of the collector illustrated by the line B is shown in FIG. 4.

FIG. 4 schematically illustrates different shape of chamber inlet ports. The figure illustrates the distal outer wall portion 13 and the radially outward extending top wall 24. The flow restrictor 29 partly blocking the end of the proximal inner wall portion 21 and the flow restrictor 29 comprises two symmetrically arranged end openings 41. The chamber inlet port 31 is illustrated as an annular shaped opening or as openings 32 shaped as a sector of an annular shape. The annular sector-shaped openings 32 are divided into sectors by supporting arms 33 extending between the outer wall and the top wall 24.

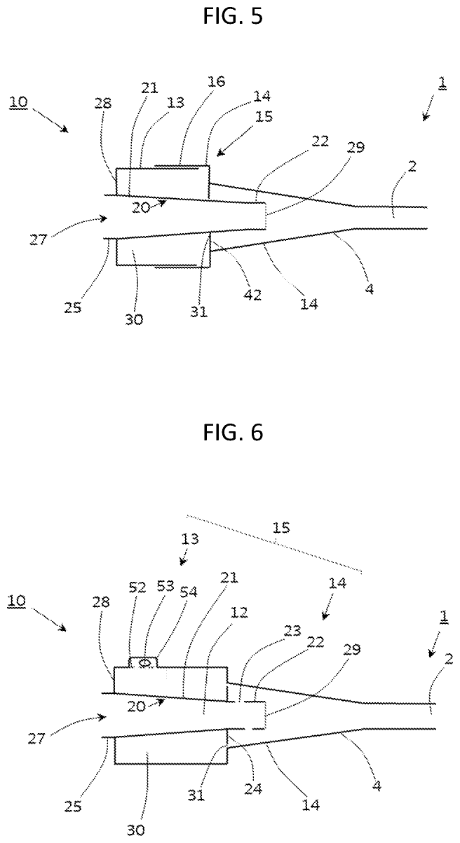

FIG. 5 illustrates an embodiment of the collector. The outer wall 15 comprises a distal outer wall portion 13, a proximal outer wall portion 14, and an overlapping outer wall portion 16. The inner wall 20 comprises a distal inner wall portion 21 which is inclined. The distal inner wall portion 21 has a frusto-conical shape.

The clearance between the inwardly extending top wall portion 42 and the outer wall may be changed as the overlapping outer wall portion 16 is displaced in the longitudinal direction of the collector. The movement of the overlapping outer wall portion 16 in longitudinal direction provides a change in the gap between the inclined distal inner wall portion and the top wall option 42. The top wall portion 42 may be adapted to open and close the one or more inlet ports 31 of the collector. The outer wall 15 may be provided with a threaded connection to allow a displacement of the overlapping outer wall portion 16 in longitudinal direction.

FIG. 6 illustrates a schematic cross-sectional side view of an embodiment of a collector and a distal part of an intermittent urinary catheter.

The proximal outer wall portion 14 is adapted to connect to/or being part of a connector of a catheter.

The catheter 1 comprises a catheter tube with a catheter lumen 2. The collector 10 has an inner wall 20, an outer wall 15, a distal end 28 and an inner lumen 12 extending from the inlet to the outlet 27. The inner lumen 12 of the collector is a continuation of the catheter lumen 2 adapted to drain fluid from the tip of the catheter and out the collector outlet.

The inner wall 20 comprises a distal inner wall portion 21, a proximal inner wall portion 22 and a top wall 24 extending radially outwards. The collecting chamber 30 is defined by the outer wall 15, the distal inner wall portion 21 and the radially outwardly extending top wall 24. Fluid may flow into the collecting chamber through the annularly shaped inlet port 31. The size and shape of the chamber inlet port 31 is defined by the small clearance between the top wall 24 and the outer wall 15.

The proximal inner wall portion 22 comprises drainage openings 23, which allows flow of fluid through the collector from a volume at the proximal end outside the proximal inner wall portion 22 and radially inwards into the intermediate portion of the lumen 12.

The collector 10 comprises a flow restrictor 29. The flow restrictor 29 may partially or completely block the proximal end of the proximal inner wall portion 22.

The collecting chamber 30 is provided with a distal outer wall portion having a weakened wall portion 52 and an adjacent positioned storage chamber 54 allowing a pellet or chemical compound/substance 53 to be pressed through the weakened wall portion 54 into the collecting chamber 30. The pellet or chemical compound/substance 53 may be dry stored in the storage chamber 54 until use in the collecting chamber.

Although particular features have been shown and described, it will be understood that they are not intended to limit the claimed invention, and it will be made obvious to those skilled in the art that various changes and modifications may be made without departing from the spirit and scope of the claimed invention. The specification and drawings are, accordingly to be regarded in an illustrative rather than restrictive sense. The claimed invention is intended to cover all alternatives, modifications, and equivalents.

1. A urine sample collector configured for collecting a sample of urine during catherization with a urinary catheter, the collector comprising
a proximal end opposite a distal end,
an inlet positioned at the proximal end and an outlet configured for drainage of fluid positioned at the distal end,
a lumen extending in longitudinal direction of the collector from the inlet to the outlet along a central longitudinal axis (A), the lumen forming a channel configured for transport of the fluid from the inlet to the outlet,
an outer wall, an inner wall and a distal end of the collector connected to the inner wall and the distal wall, with the outer wall encircling the inner wall, the inner wall defining an inner volume measured at a distal part of the collector corresponding to a distal part of the lumen,
wherein the outer wall extends longitudinally to provide a distal outer wall portion and a proximal outer wall portion, and the inner wall comprising a distal inner wall portion,
wherein the distal outer wall portion, the distal inner wall portion and the distal end defines a collecting chamber, wherein the collecting chamber comprises one or more chamber inlet ports, the one or more chamber inlet ports being defined by a clearance between the outer wall and the inner wall.

2. The collector according to claim 1, wherein the one or more chamber inlet ports comprises an annularly shaped opening wall between the outer wall and the inner wall.

3. The collector according to claim 1, wherein the distal outer wall portion and the distal inner wall portion are co-axial in the longitudinal direction along the central longitudinal axis (A) of the collector.

4. The collector according to claim 1, wherein the one or more chamber inlet ports are radially offset from the central longitudinal axis (A), and the one or more chamber inlet ports extend in a radial direction between the inner wall and the outer wall and are positioned facing in longitudinal direction towards the proximal end of the collector.

5. (canceled)

6. The collector according to claim 1, wherein the inner wall comprises a top wall, which extends outwardly in radial direction towards the outer wall, where the radial extent of the one or more chamber inlet ports is defined by the clearance between the top wall and the outer wall in the radial direction.

7. The collector according to claim 1, wherein the outer wall comprises a top wall portion, which extends inwardly in radial direction towards the inner wall, where a size of the one or more chamber inlet ports is defined by the clearance between the top wall portion and the inner wall in the radial direction.

8. (canceled)

9. The collector according to claim 1, further comprising:
a flow restrictor positioned in the lumen and adapted to disrupt axial flow of the liquid to the outlet and to direct a portion of the liquid to the one or more chamber inlet ports.

10. The collector according to claim 1, wherein the inner wall comprises a proximal inner wall portion defining an inner volume at an intermediate portion of the lumen, where the intermediate portion of the lumen interconnects the proximal portion of the lumen and the distal portion of the lumen, the proximal portion and the intermediate portion and the distal portion of the lumen forming a through-going passage for providing flow of liquid through the collector.

11. The collector according to claim 10, wherein the proximal inner wall portion comprises one or more side drainage openings allowing flow of fluid from a volume outside the proximal inner wall portion and radially inwards into the intermediate portion of the lumen.

12. A The collector according to claim 10, wherein the collector comprises at least one end opening at the proximal end of the proximal inner wall portion, where the at least one end opening extends in radial and or axial direction adapted to allow passage of fluid into the intermediate portion of the lumen through the proximal end or side wall of the proximal inner wall portion.

13. The collector according to claim 12, wherein the total size of the one or more side drainage openings and the at least one end opening is equal to or larger than the smallest cross-sectional area of the inlet.

14. The collector according to claim 1, wherein the lumen comprises a varying cross-sectional area in a radial direction, where a smallest size of the cross-sectional area is equal to or larger than a size of the cross-sectional area of the inlet.

15. The collector according to claim 1, wherein the one or more chamber inlet ports comprises a cross-sectional size in a transverse direction in a range of 20-60% of a size of a largest cross-sectional area of the distal outer wall portion.

16. The collector according to claim 1, wherein the collecting chamber comprises two or more sub-chambers.

17. The collector according to claim 1, wherein the proximal outer wall portion comprises a coupling port adapted to connect the one or more chamber inlet ports of the collector to a distal end of a catheter lumen.

18. The collector according to claim 1, wherein the proximal outer wall portion and the distal outer wall portion comprises a flap adapted to open a gate of the one or more inlet ports.

19. The collector according to claim 1, wherein the distal outer wall portion comprises a see-through portion adapted for a user to see through the outer wall.

20. The collector according to claim 1, wherein the collecting chamber further comprises a test pad.

21. The collector according to claim 1, wherein the distal outer wall portion further comprises an access port adapted to allow a pellet of a chemical compound to be deposited into the collecting chamber.

22. The collector according to claim 1, wherein the collector further comprises a gateway adapted for extracting a sample of urine from the collecting chamber.

23. An intermittent urinary catheterisation assembly for self-catheterisation comprising:
a catheter tube comprising an insertable portion intended for insertion into a urethra, where the catheter tube comprises a catheter lumen and an outer surface of the insertable portion of the catheter tube comprises a hydrophilic surface coating, and a connecting portion coupled with the catheter tube; and the collector according to claim 1 attachable to the connecting portion of the catheter tube.

24. The intermittent urinary catheterisation assembly according to claim 23, wherein the inner lumen of the urine sample collector and the catheter lumen of the catheter tube are so proportioned that a cross-sectional area of the inner lumen of the collector is equal to or greater than a cross-sectional area of the catheter lumen of the catheter.

25. (canceled)
